(19) United States
(12) Patent Application Publication
(10) Pub. No.: US 2026/0272637 A1
(43) Pub. Date: Sep. 17, 2026

(54) REINFORCED MEDICAL IMPLANT AND METHOD OF USE

(71) Applicants: Smith & Nephew, Inc., Memphis, TN (US); Smith & Nephew Asia Pacific Pte. Limited; Smith & Nephew Orthopaedics AG, Zug (CH)

(72) Inventors: Collin W. Shale, Massachusetts, MA (US); Helawe Betre, Austin, TX (US)

(73) Assignees: Smith & Nephew, Inc., Memphis, TN (US); Smith & Nephew Asia Pacific Pte. Limited, (omitted); Smith & Nephew Orthopaedics AG, Zug (CH)

(21) Appl. No.: 19/674,870

(22) Filed: May 12, 2026

Related U.S. Application Data
(63) Continuation of application No. PCT/US2024/057236, filed on Nov. 25, 2024.
(60) Provisional application No. 63/602,765, filed on Nov. 27, 2023.

(51) Int. Cl. A61F 2/08; A61F 2/00

(52) U.S. Cl. CPC A61F 2/08; A61F 2002/0068; A61F 2002/0081

(57) ABSTRACT

A bio-compatible implant that is adapted for use in repairing soft tissue damage includes a fibrous scaffold having a length, a width and a thickness. The fibrous scaffold includes a plurality of fibers oriented in three orthogonal directions. In some instances, the three orthogonal directions align with the length, width and thickness of the fibrous scaffold. In some cases, two of the three orthogonal directions may be offset with respect to the length and width of the fibrous scaffold.

### CROSS REFERENCE TO RELATED APPLICATION

This application is a continuation of International Application No. PCT/US2024/057236, filed Nov. 25, 2024, which claims priority to U.S. Provisional Application No. 63/602,765, filed Nov. 27, 2023, which are herein incorporated by reference in their entirety.

### TECHNICAL FIELD

The present disclosure pertains generally, but not by way of limitation, to orthopedic implants and methods of treatment. More particularly, the present disclosure relates to a tendon repair implant, such as one that is engineered for arthroscopic placement over or in the area of a full or partial thickness tear of the supraspinatus tendon of the shoulder.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Adequate procedures do not exist for repairing a partial thickness tear of less than 50% in the supraspinatus tendon. Current procedures attempt to alleviate impingement or make room for movement of the tendon to prevent further damage and relieve discomfort but do not repair or strengthen the tendon. Use of the still damaged tendon can lead to further damage or injury. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for implants used for soft tissue repairs such as but not limited to rotator cuff repairs. An example may be found in a bio-compatible implant adapted for use in repairing soft tissue damage. The bio-compatible implant may include a fibrous scaffold having a longitudinal axis. The fibrous scaffold may include a plurality of fibers arranged in a three dimensional pattern in order to strengthen the fibrous scaffold against forces applied to the fibrous scaffold, including forces that are applied along the major axis. The three dimensional pattern may include fibers oriented in three orthogonal directions. The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a cross-section of an anterior view of a shoulder of a patient;

FIG. 2 illustrates an example bio-compatible implant;

FIG. 3 illustrates a portion of an example fibrous scaffold that may form part of the example bio-compatible implant of FIG. 2;

FIG. 4 illustrates a portion of the example fibrous scaffold of FIG. 3;

FIG. 5 illustrates a portion of the example fibrous scaffold of FIG. 3;

FIG. 6 illustrates a portion of the example fibrous scaffold of FIG. 3;

FIG. 7 illustrates a portion of an example fibrous scaffold that may form part of the example bio-compatible implant of FIG. 1;

FIG. 8 illustrates a portion of the example fibrous scaffold of FIG. 7;

FIG. 9 illustrates a portion of the example fibrous scaffold of FIG. 7; and

FIG. 10 illustrates a number of example three dimensional printing patterns.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current repair procedures may attempt to alleviate impingement or make room for movement of the tendon to prevent further damage and relieve discomfort but do not repair or strengthen the tendon. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

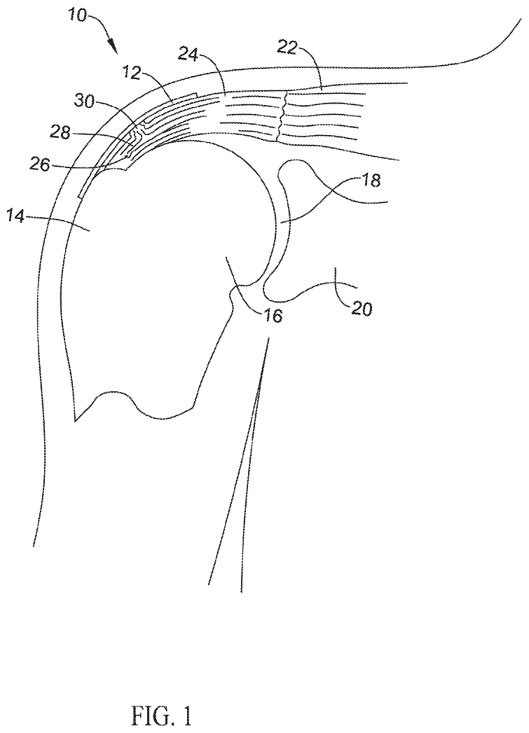

FIG. 1 shows a cross-sectional view of a shoulder 10 including an example implant 12. Shoulder 10 further shows a head 14 of a humerus 16 mating with a glenoid fossa 18 of scapula 20. The glenoid fossa 18 includes a shallow depression in scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1, the distal tendon 24 includes a damaged portion 28 located near the insertion point 26. The damaged portion 28 includes a tear 30 extending partially through the distal tendon 24. The tear 30 may be referred to as a partial thickness tear. The depicted partial thickness tear 30 is on the bursal side of the tendon, however, the tear may also be on the opposite or articular side of the distal tendon 24 and/or may include internal tears to the distal tendon 24 not visible on either surface. In other instances the tear 30 may be a full thickness tear.

FIG. 1 further illustrates that the tendon repair implant 12 has been placed over the partial thickness tear 30. In this example, the tendon repair implant 12 is placed on the bursal side of the tendon regardless of whether the tear is on the bursal side, articular side or within the tendon. Further, the tendon repair implant 12 may overlay multiple tears.

Although one possible treatment site is described in the figures as being located in a shoulder joint, the implant 12 may be used at a variety of different treatment sites, such as the hip, knee, ankle, et. Furthermore, the implant 12 may be used for any of a variety of soft tissue repairs, such as but not limited to the Gluteus Medius, which is a large fan-shaped muscle located in the posterior hip, the Hip Capsule, which is also in the hip. The implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The implant 12 may be used in treating soft tissue injuries in the Achilles tendon. The implant 12 may be used in treating soft tissue injuries for any areas of the body that will accommodate the implant 12.

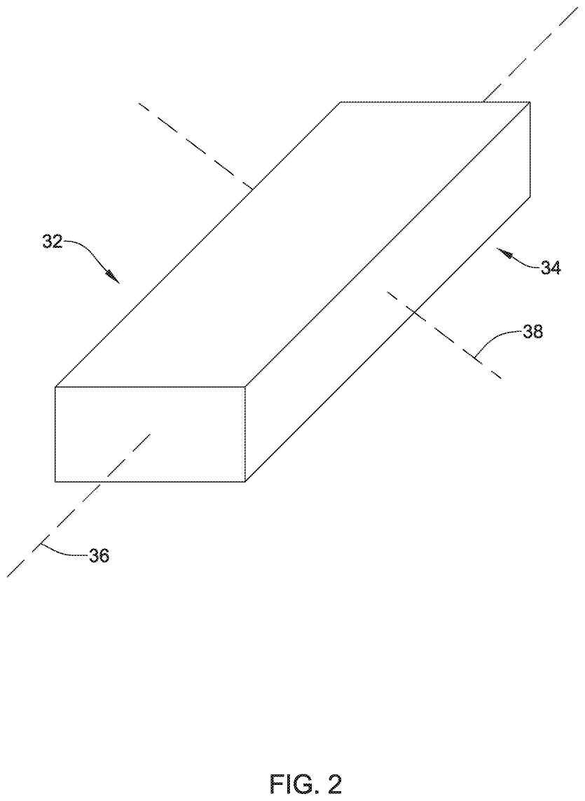

FIG. 2 is a perspective view of an example bio-compatible implant 32 that is adapted for use in repairing soft tissue damage. The bio-compatible implant 32 may be considered as being an example of the tendon repair implant 12 shown with respect to FIG. 1. In some cases, the bio-compatible implant 32 may be considered to be adapted for repairing damage to a rotator cuff or to rotator cuff tendons. In some cases, the bio-compatible implant 100 may be considered to be adapted for use in repairing damaged tendons in other parts of the body, such as but not limited to the knee and the hip.

The bio-compatible implant 32 includes a fibrous scaffold 34. While the fibrous scaffold 34 is shown as being rectilinear in shape, this is just an example. In some instances, the fibrous scaffold 34 may take any of a variety of different shapes, for example. The fibrous scaffold 34 may be considered as having a major axis 36 that is aligned with a long direction of the fibrous scaffold 34 and a minor axis 38 that is orthogonal to the major axis 36. In some instances, the fibrous scaffold 34 may have a length in a direction along the major axis 36 that is in a range of about 10 to about 50 centimeters. The fibrous scaffold 34 may have a width in a direction along the minor axis 38 that is in a range of about 5 centimeters to about 30 centimeters, for example. The fibrous scaffold 34 may have a thickness in a direction orthogonal to both the major axis 36 and the minor axis 38 that is in a range of about 0.5 millimeters to about 4 millimeters. These dimensions are merely illustrative.

In some cases, the fibrous scaffold 34 may be formed from any of a variety of different fibrous materials. In some cases, the fibrous scaffold 34 may be formed of collagen. In some cases, the fibrous scaffold 34 is formed of collagen that has been dehydrated. The fibrous scaffold 34 may be formed as a fibrous collection of collagen fibers that define a porous scaffold with a large number of void spaces.

The collagen used to form the fibrous scaffold 34 may come from any of a variety of different sources. Collagen is a main structural protein in the extracellular matrix found in various connective tissues and thus can be obtained from various animals. For example, the collagen used to form the fibrous scaffold 34 may be bovine-based, i.e., from cows. In some cases, the collagen used to form the fibrous scaffold 34 may come from bovine tendon material. As an illustrative but non-limiting example, bovine Achilles tendon is digested down to highly purified collagen, dissolved and/or dispersed into an acid solution and extruded into a neutralizing fluid bed. The fibrous scaffold 34 may be formed of collagen that has been obtained in other methods as well.

While the fibrous scaffold 34 may be formed having a variety of different porosity levels (defined as relative amount of void space to solid material), in some cases the fibrous scaffold 34 may have a porosity of at least 50 percent or more, at least 60 percent or more, at least 70 percent or more, or at least 80 percent or more. In some cases, the fibrous scaffold 34 may have a porosity of 60 percent to 90 percent, 70 percent to 90 percent, 80 percent to 90 percent, or 85 percent to 90 percent.

Another way to define the fibrous scaffold 34 is in terms of average pore size. Pore size refers to a diameter of voids, or empty spaces, formed within the fibrous scaffold 102. Average pore size, accordingly, refers to an average diameter of these voids. In some cases, the fibrous scaffold 34 may have an average pore size of 20 microns or greater, or 30 microns or greater, or 40 microns or greater, or 50 microns or greater, or 60 microns or greater, or 70 microns or greater, or 80 microns or greater, or 90 microns or greater. In some cases, the fibrous scaffold 34 may have an average pore size that is in a range of 100 microns to 500 microns, 100 microns to 400 microns, 100 microns to 300 microns, 100 microns to 150 microns, or 200 microns to 400 microns, for example.

In some cases, the fibrous scaffold 34 may include a plurality of fibers that are not randomly oriented, but rather are oriented in two or more orthogonal directions. In some cases, these two or more orthogonal directions align with the major axis 36, the minor axis 38 and/or the thickness of the fibrous scaffold 34. In some cases, depending on how the bio-compatible implant 32 including the fibrous scaffold 34 is used, the major axis 36 may be parallel with a Lateral-Medial designation. The minor axis 38 may be parallel with a Superior-Inferior designation. In some cases, the layers 42, 44, 46 may be stacked in a direction corresponding to an Anterior-Posterior-Anterior direction.

In some cases, the two or more orthogonal directions do not align with each of the major axis 36 and the minor axis 38, but instead may be disposed at a an angle relative to the major axis 36 and/or the minor axis 38. In some cases, the fibrous scaffold 34 may include lattice elements that are formed by a collection of collagen fibers that are aligned along a path of the lattice. In some cases, the lattice elements may be interlaced with each other in a three dimensional, periodic pattern that repeats itself in multiple dimensions. Locking members, which can be fibers, may run transverse to a primary loading axis such that tensioning along the long axis engages the locking members. As an example, the lattice elements may have a diameter that ranges from 0.03 millimeters to 0.08 millimeters.

Three dimensions (3D) printing with a solution including collagen fibers may be used to form fibers that are oriented in a desired orientation relative to other fibers and/or relative to the fibrous scaffold 34 itself. In some cases, 3D printing with a high collagen concentration solution may be deposited along a print path using continuous extrusion at speeds exceeding 10 meters per second using a fine gauge needle (i.e., 27G or 32G needle) into an environmentally controlled fluid bed that neutralizes and facilitates stabilization and fibrilization of the extruded collagen slurry. A variety of different three dimensional patterns for the fibers within the fibrous scaffold 34 may be achieved.

In some cases, multiple extrusion needles may be used, such as one extrusions needle for each of the orthogonal directions. In some cases, the collagen is allowed to fibrilize to a selected pattern by changing support bed environment such as pH. The environmentally controlled fluid bed may be removed by changing temperature, and the printed structure may be stabilized by drying and exogenous crosslinking using materials such as one or more of formaldehyde, glutaraldehyde, carbodiimide, and others. In some cases, the collagen fibrils align along the printing path. This printing process, which may be referred to as an embedded bioprinting method, and is available from the vendor FluidForm. In some cases, the fibrous scaffold 34 may include not only collagen fibers, but may also include fibers formed from other polymeric materials.

When printing with a high collagen concentration solution (e.g., greater than 10 milligrams per milliliter), the resulting fibers are composed of individual collagen fibers that are bonded to each other to form a continuous collagen fiber. One example of a three dimensional fiber pattern is a pseudo weave pattern in which the three orthogonal directions of the pseudo weave pattern may align with the major axis 36, the minor axis 38 and the thickness dimension. In some cases, the pattern includes a plurality of fibers including a first plurality of fibers that extend along the length of the fibrous scaffold 34 within each of a plurality of layers stacked along the thickness of the fibrous scaffold 34, each of the first plurality of fibers within each layer following a periodic wave pattern along the length of the fibrous scaffold 34 defining alternating peaks and troughs in which the fiber alternates between a first direction orthogonal to the length and a second direction orthogonal to the length, the fiber remaining within a single layer. In some cases, the periodic wave pattern may be an oscillatory wave pattern. In some cases, the periodic wave pattern may be a sinusoidal wave pattern. In some cases, the periodic wave pattern may be a square wave pattern. In some cases, the fibrous scaffold 34 may include fibers arranged according to more than one different type of wave pattern.

In some cases, each fiber of the first plurality of fibers within a layer may be laterally offset from neighboring fibers within that layer. In some cases, each fiber of the first plurality of fibers within a layer may overlap with neighboring fibers in an adjacent layer. Each fiber may define peaks that overlap with peaks and troughs that overlap with troughs of neighboring fibers in an adjacent layer. In some cases, the fibrous scaffold 34 may further include a second plurality of fibers that extend orthogonally to the first plurality of fibers and extend through multiple layers. Some of the second plurality of fibers may extend through peaks formed by fibers of the first plurality of fibers within a plurality of layers and some of the second plurality of fibers may extend through troughs formed by fibers of the first plurality of fibers within a plurality of layers. In some cases, at least two fibers of the second plurality of fibers extend through each of the peaks and through each of the troughs. In some cases, there may be only one fiber of the second plurality of fibers, or even three or more fibers of the second plurality of fibers, that extend through each of the peaks and each of the troughs. In some cases, the number of fibers and relative spacing may be adjusted to potentially control the amount of stretch that the fibrous scaffold will undergo during initial loading.

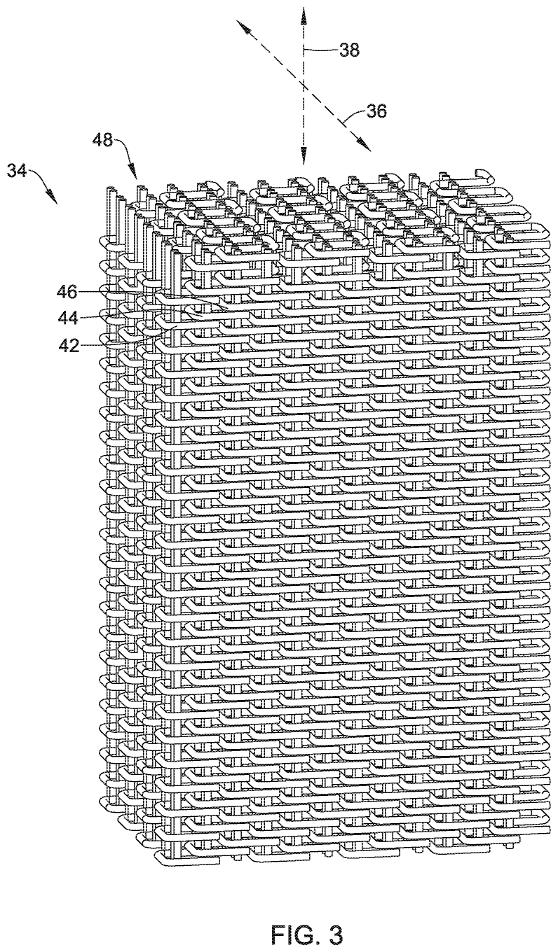

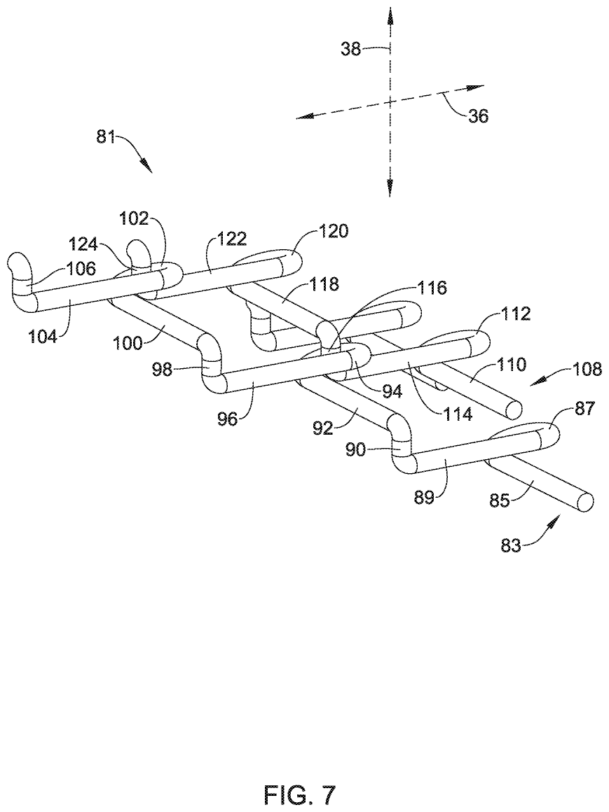

A sample pseudo weave pattern is shown for example in FIGS. 3-6. Another example of a three dimensional fiber pattern is a knit pattern in which at least two orthogonal directions of the knit pattern may be offset relative to the major axis 36 and/or the minor axis 38. A sample knit pattern is shown for example in FIGS. 7-9. These patterns are merely illustrative, as any of a variety of different three dimensional fiber patterns may be created.

FIG. 3 illustrates a portion of an example fibrous scaffold 40 that may be considered as an example of a pseudo weave pattern. The fibrous scaffold 40 may be considered as being an example of the fibrous scaffold 34 that is schematically shown in FIG. 2. As illustrated, the major axis 36 extends front to back, the minor axis 38 extends top to bottom, and the thickness of the fibrous scaffold 40 extends right to left. It can be see that the fibrous scaffold 40 may be considered as including a number of layers in which the pattern repeats. As an example, consider a layer 42, a layer 44 and a layer 46. A close review of the fibrous scaffold 40 reveals that these three layers 42, 44 and 46 may be considered as repeating top to bottom within the fibrous scaffold 40. While the fibrous scaffold 40 shows a repeating pattern (top to bottom) of three repeating layers 42, 44, and 46, it will be appreciated that the fibrous scaffold 40 may include four or more distinct repeating layers. In some cases, the fibrous scaffold 40 may include only two distinct repeating layers. In some cases, the fibrous scaffold 40 may be considered as including a single repeating layer that is offset in one direction a limited number of times, and then offset in an opposing direction a limited number of times before reverting again. In some cases, the fibrous scaffold 40 may be considered as including a single repeating layer that is repeatedly offset enough times to create a desired thickness for the fibrous scaffold 40 if considering the bulk of the fibrous scaffold 40. Obviously, along the perimeter of the fibrous scaffold 40, the repeating pattern may be interrupted.

As will be discussed, each of the repeating layers 42, 44 and 46 are actually the same, but are laterally offset relative to one another. Each of the repeating layers 42, 44, 46 include fibers that extend longitudinally, along the major axis 36, but also undulate back and forth in a sinusoidal pattern forming peaks and troughs. A peak involves a fiber first extending in a first direction along the minor axis 38, then extending along the major axis 36, then extending in a second (opposing) direction along the minor axis 38. A trough involves a fiber first extending in the second direction along the minor axis 38, then extending along the major axis 36, then extending in the first direction along the minor axis 38. The peaks and troughs may be considered as being coplanar within the repeating layer 42, 44, and 46.

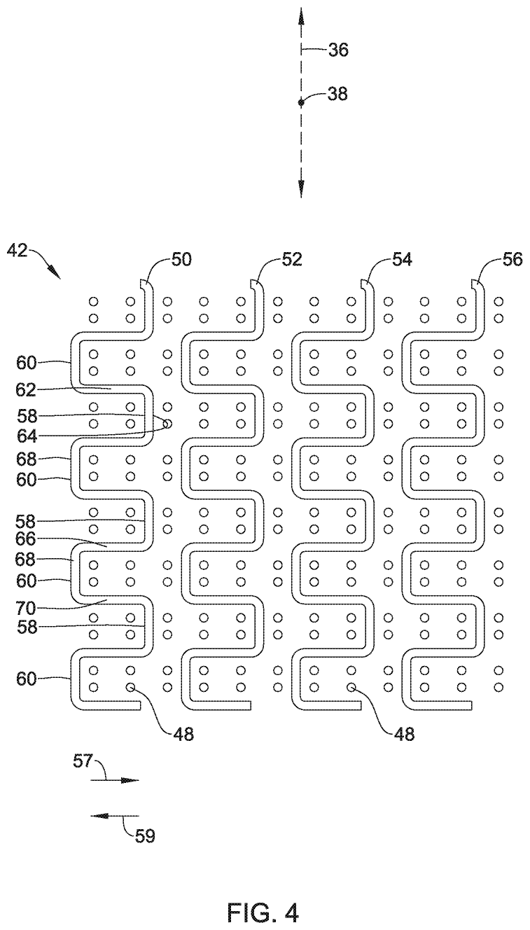

FIG. 4 is a schematic view of the repeating layer 42. As shown, the repeating layer 42 includes a first plurality of fibers including a fiber 50, a fiber 52, a fiber 54 and a fiber 56 that extend through the layer 42 in a direction aligning with the major axis 36. While only four fibers are shown, this is merely illustrative as the layer 42 will likely include substantially more than four fibers 50, 52, 54, and 56. It will be appreciated that each of the fibers 50, 52, 54, and 56 have the same profile. Looking at the fiber 52, it can be seen that the fiber 52 forms several peaks 58 and several troughs 60, all of which lie within a plane defined by the layer 42. For a particular peak 58, the fiber 52 has a portion 62 that extends in a first direction indicated by an arrow 57, a portion 64 that extends parallel with the major axis 36 and a portion 66 that extends in a second direction indicated by an arrow 59. For an adjacent trough 60, the fiber 52 has a portion 66 that extends in the direction indicated by the arrow 59, a portion 68 that extends parallel with the major axis 36 and a portion 70 that extends in the direction indicated by the arrow 57. Obviously, the portion 66 defines a leg of the peak 58 as well as a leg of the adjoining trough 60. A second plurality of fibers 48 may be seen as extending through the layer 42, orthogonally to both the major axis 36 and the minor axis 38. The fibers 48 may be considered as extending through a thickness of the fibrous scaffold 34, for example. The fibers 48 and the offset of layers helps with coupling each layer to each other mechanically.

In some cases, the fibers forming the repeating layer 42, the repeating layer 44 and the repeating layer 46 may vary in profile and three dimensional profile. As an example, the sinusoidal pattern may have as many as six or more variations, can be implemented in separate layers by adjusting one or more parameters such as phase angle, short-axis offset, or by inverting the amplitude of the sinusoid. Each layer 42, 44 and 46 may be 3D printed, in combination with the segment of each of the fibers 48 that extend through that particular layer. Once a layer 42 is printed, the layer 44 may be printed, and then the layer 46 may be printed, and so on. After all of the layers have been printed, each of the fibers 48 may be printed. The needle may be placed in the location of the orthogonal fiber at the deepest layer, and a particular fiber 48 is printed in a single go with a straight, z-axis pull. The needle may then be moved to another location at the deepest layer, and the process may be continued until all of the fibers 48 have been printed.

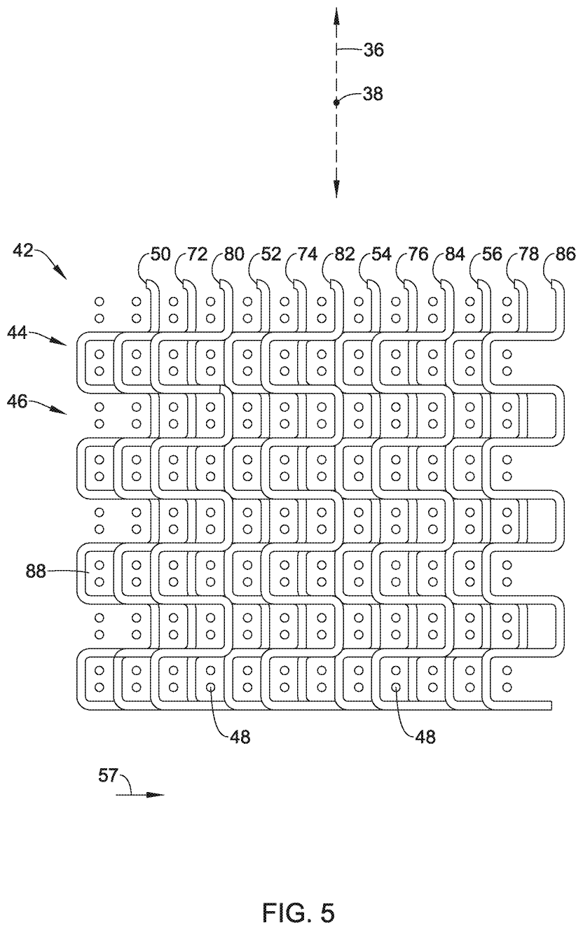

FIG. 5 is a schematic view of the repeating layer 42, the repeating layer 44 and the repeating layer 46, with the layers 42, 44 and 46 disposed atop one another. It will be appreciated that the repeating layer 44 is a replica of the repeating layer 42, just offset in a direction indicated by the arrow 57 and the repeating layer 46 is a replica of the repeating layer 42, just offset further in a direction indicated by the arrow 57. The layer 42 may be seen as including the fibers 50, 52, 54 and 56, as described with respect to FIG. 4. The layer 44 includes fibers 72, 74, 76 and 78, each of which extend in a direction parallel with the major axis 36 while undulating back and forth within the layer 44, in alternating directions parallel with the minor axis 38 (as shown in FIG. 4 with respect to the fiber 50). The layer 46 includes fibers 80, 82, 84, and 86, each of which extend in a direction parallel with the major axis 36 while undulating back and forth within the layer 44, in alternating directions parallel with the minor axis 38 (as shown in FIG. 4 with respect to the fiber 50). Because the layer 44 is offset from the layer 42, and the layer 46 is offset from the layer 44, the fiber 72 (part of the layer 44) is offset from the fiber 50 (part of the layer 42), and the fiber 80 (part of the layer 46) is offset from the fiber 72, and so on.

As noted, the second plurality of fibers 48 extend through the layers 42, 44, 46. Consider a segment 88 defined between the fiber 50 and the fiber 72. As can be seen, two fibers 48 extend through the segment 88 but do not touch either the fiber 50 or the fiber 72. It will be appreciated that these segments extend throughout the fibrous scaffold 34, with the fibers 48 not touching any of the fibers forming the segments. When a tensile force is applied along the major axis 36, the fibers 48 limit relative movement of the fibers extending orthogonally to the fibers 48 and thereby provide additional resistance to the applied tensile force.

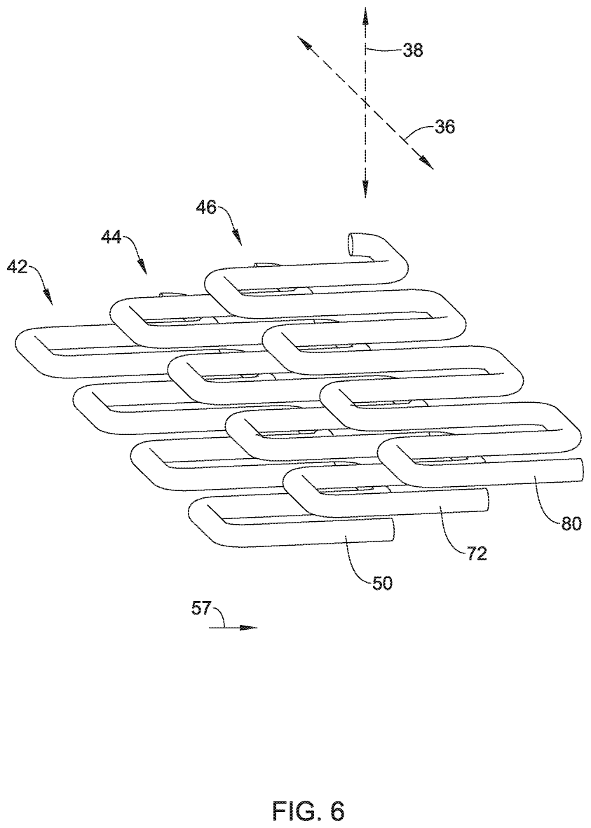

FIG. 6 is a perspective view showing how the layer 44 is stacked atop the layer 42 and how the layer 46 is stacked atop the layer 44. For clarity, the fibers 48 are not shown. FIG. 6 also shows the fiber 72, forming part of the layer 44, is laterally offset in a direction indicated by the arrow 57 relative to the fiber 50 forming part of the layer 42. FIG. 6 also shows how the fiber 80, forming part of the layer 46, is laterally offset in a direction indicated by the arrow 57 relative to the fiber 72 forming part of the layer 44, and is further laterally offset in a direction indicated by the arrow 57 relative to the fiber 50 forming part of the layer 42. In forming the fibrous scaffold 40, the fibers within a particular layer may be printed. After a particular layer is formed, the layer above that particular layer may be printed.

In some cases, forming the fibrous scaffold 40 may involve a first stage of “weft” printing and a second stage of “warp” printing. During “weft” printing, periodic structures are printed along the primary loading access (parallel to the major axis 36) until a desired thickness is achieved. A new “slice” of periodic structure is printed, offset in the thickness axis. During “warp” printing, the transverse members are printed between weft offsets. The transverse members are repeated to fill all openings.

It should be noted that while certain segments of the fibers making up the fibrous scaffold 40 are described and shown as being straight, this is not required in all cases. For example, at least some of the straight segments may curve in one or more planes. While certain segments are described as being orthogonal to each other, in some cases some of the segments may not be orthogonal, but may cross each other at acute angles that are less than ninety degrees.

FIGS. 3 through 6 provide views of the fibrous scaffold 40 in which the two or more orthogonal directions in which fibers are orientated align with the major axis 36, the minor axis 38 and/or the thickness of the fibrous scaffold 34. In some cases, two of the three orthogonal directions of the fibrous scaffold may instead be offset relative to the major axis 36 (length) and the minor axis 38 (width) of the fibrous scaffold. FIGS. 7 through 9 provide various views of a fibrous scaffold 81 in which the fibers making up the fibrous scaffold 80 are offset relative to the major axis 36 and the minor axis 38. The fibrous scaffold 81 may be considered as an example of the fibrous scaffold 34 shown in FIG. 2.

In some cases, the plurality of fibers making up the fibrous scaffold 81 may each include multiple periods of a loop pattern in which each fiber repeatedly includes a climbing loop, a vertical segment and a straight segment joining the climbing loop and the vertical segment. For each of the plurality of fibers, the vertical segment may pass through a climbing loop of another fiber. In some cases, the straight segment after a climbing loop may extend in a direction orthogonal to a direction in which the straight segment before the climbing loop extends. In some cases, the straight segment after a climbing loop may also be offset in a direction orthogonal to the direction in which the straight segment after the climbing loop extends and orthogonal to the direction in which the straight segment before the climbing loop extends. In some cases, each vertical segment may extend in a direction orthogonal to the direction in which the straight segment after the climbing loop extends and orthogonal to the direction in which the straight segment before the climbing loop extends.

FIG. 7 is a perspective view of a portion of the fibrous scaffold 81. Individual fibers within the fibrous scaffold 81 are not contained within a single layer, but rather individual fibers within the fibrous scaffold 81 extend in a first direction that may align with either of the major axis 36 or the minor axis 38, may extend through a climbing loop, a short straight segment and a vertical segment. In some cases, the individual fibers within the fibrous scaffold 81 may also be offset relative to the major axis 36 and the minor axis 38. To illustrate, the fibrous scaffold 81 includes a fiber 83. As shown, the fiber 83 has a straight segment 85, a climbing loop 87 that includes a vertical component such that the straight segment 89 that extends from the climbing loop 87 is able to cross over the straight segment 85 and extend orthogonally to the straight segment 85. The fiber 83 has a vertical segment 90 that leads to a straight segment 92. A climbing loop 94 includes a vertical component such that a straight segment 96 extending from the climbing loop 94 is able to cross over the straight segment 92 and extend orthogonally to the straight segment 92. The fiber 83 has a vertical segment 98 that leads to a straight segment 100. A climbing loop 102 includes a vertical component such that a straight segment 104 extending from the climbing loop 102 is able to cross over the straight segment 100 and extend orthogonally to the straight segment 102. As shown, the fiber 83 terminates in a vertical segment 106. It will be appreciated that the fiber 83 may continue on well beyond the vertical segment 106, depending on relative position within the fibrous scaffold 81.

The fibrous scaffold 81 includes a fiber 108. As shown, the fiber 108 has a straight segment 110, a climbing loop 112 that includes a vertical component such that a straight segment 114 may cross over the straight segment 110 and may extend orthogonally to the straight segment 110. The fiber 108 includes a vertical segment 116 that extends through the climbing loop 94 formed by the fiber 83. From the vertical segment 116, the fiber 108 includes a straight segment 118 and a climbing loop 120 that includes a vertical component such that a straight segment 122 is able to cross over the straight segment 118 and extends orthogonally to the straight segment 118. As shown, the straight segment 122 terminates in a vertical segment 124 that extends through the climbing loop 102 formed by the fiber 83. It will be appreciated that the fiber 108 may continue well beyond the vertical segment 124, depending on relative position within the fibrous scaffold 80.

In some cases, none of the fibers actually touch each other, and instead are spaced apart. Even the vertical segments extending through corresponding climbing loops do not touch the climbing loops. Straight segments that cross over and are orthogonal to another straight segment are spaced apart, and do not actually touch each other. It will be appreciated that FIG. 7 only shows a small fraction of the fibrous scaffold 81. The pattern shown may be repeated to attain complete coverage over one of the lattice dimensions, after which the pattern is staggered and repeated enough times to fill in the other lattice dimensions. In the completed fibrous scaffold 81, each climbing loop formed by one fiber will have a vertical segment formed by another fiber extending through the climbing loop. In that way, when a tensile force is applied along the major axis 36, the vertical segments interact with the climbing loops through which they extend, thereby limiting stretching of the fibrous scaffold 81.

It should be noted that while certain segments of the fibers making up the fibrous scaffold 81 are shown and described as straight, this is not required in all cases. For example, at least some of the straight segments may curve in one or more planes. While certain segments are described as being orthogonal to each other, in some cases some of the segments may not be orthogonal, but may cross each other at acute angles that are less than ninety degrees.

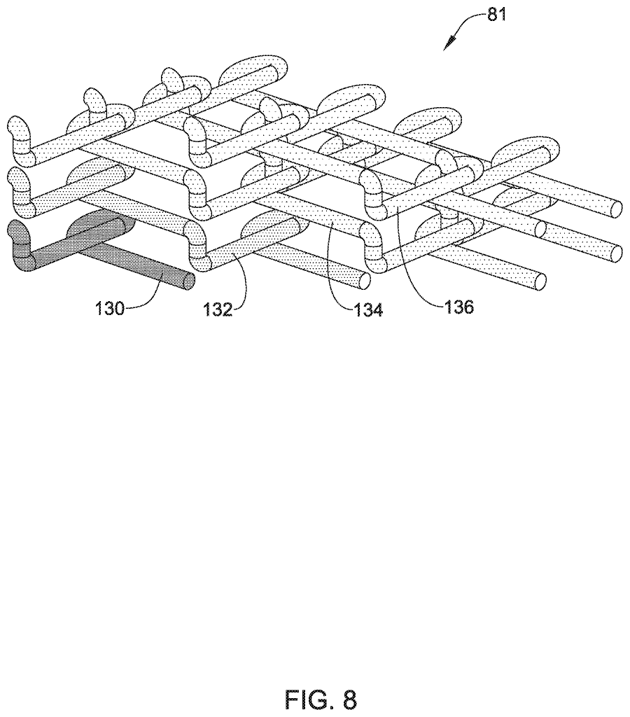

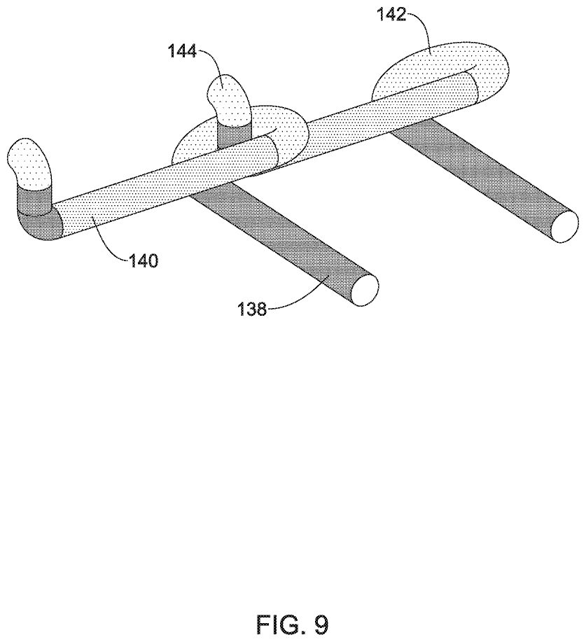

It will be appreciated that forming the fibrous scaffold 81 may be more involved than forming the fibrous scaffold 40 because the fibers forming the fibrous scaffold 81 have more of a three dimensional component while the fibers forming the fibrous scaffold 40 are in distinct layers. FIGS. 8 and 9 provide an example of how the fibrous scaffold 81 may be formed using 3D printing. FIG. 8 shows a portion of the fibrous scaffold 81, showing that at the lattice level, printing may involve starting at the lowest z-axis height, and then working towards the right and up (with respect to the illustrated orientation). A section 130 may be printed first. Next, a section 132 may be printed after the section 130 has been completed. Next, a section 134 may be printed after the section 132 has been completed. Next, a section 136 may be printed after the section 134 has been completed. While only four sections are shown, it will be appreciated that the fibrous scaffold 80 may include substantially more than four sections, depending on the overall dimensions of the fibrous scaffold 81. At a chain level, as shown in FIG. 9, printing may begin with a section 138, followed sequentially by a section 140, a section 142 and a section 144. Additional sections may be included.

In some cases, there are variables that may adjusted or otherwise controlled in order to adjust the resulting fibrous scaffold 81. For example, in defining the climbing loops exhibiting a circular helix, the X, Y and Z positions may be given by the equations:

$$X = R * cos ( t )$$

$$Y = R * sin ( t )$$

$$Z = ( offset ) * ( angle * π / 180 ) * t ,$$

where R is radius and t goes from a start time (t1) to an end time (t2). Variables that may impact formation of straight segments include length and angle. Variables that may impact the Z-step include helical radius, height and fiber diameter. Additional variables may include offset height, for example. In some cases, the climbing loops may not exhibit a circular helix, but may have other shapes. In such cases, different parametric equations are appropriate.

In some cases, the fibrous scaffold 40 and/or the fibrous scaffold 81 may be formed via 3D bioprinting. In some cases, the fibrous scaffold 40 and/or the fibrous scaffold 81 may be formed by weaving sufficiently small biofibers on specialized looms. If weaving were achieved, multiple traditional woven patterns may be attainable using this biofiber. In some cases, changes may be made to composition. For example, the fiber patterns described herein may be overprinted onto a resorbable mesh such as silk or PLLA (poly-L-lactic acid) in order to increase specific tensile properties of the construct. The density of the collagen slurry may be varied, resulting in higher or lower densities of collagen fibrils in the final construct. The transverse members such as the fibers 48 may be replaced with a resorbable polymer in order to further increase strength.

Changes in form may include the primary pattern being rectilinear with the print path aligned to the primary loading axis, with incorporated sections of the pattern shown in the fibrous scaffold 40 may be disposed around either the edges of the implant or specified areas of the implant where sutures or other fixation members may be secured as a ripstop. The primary tensile strength of the implant may be provided by the rectilinear region.

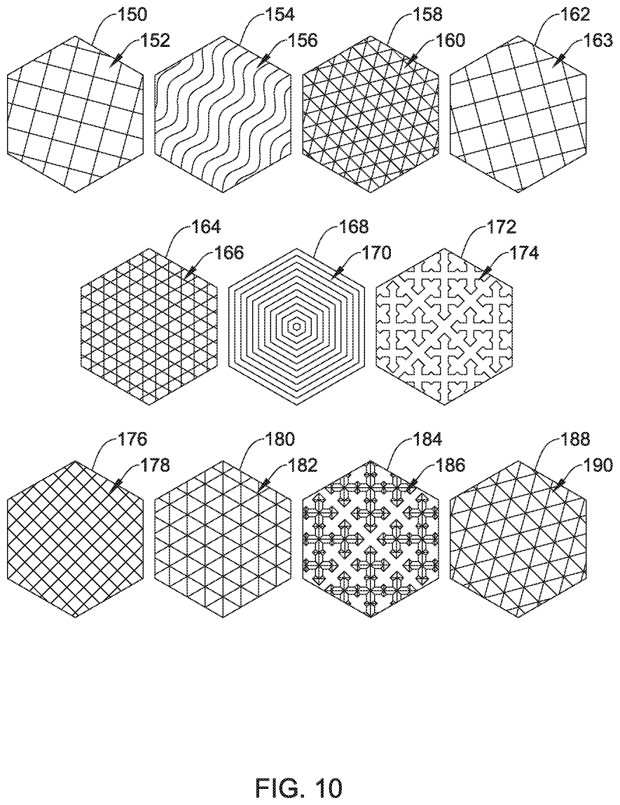

In some cases, an implant may be formed by 3D printing, and may include an outer shell consisting of a series of rectilinear print paths, with an inner core that is formed using the patterns used to form the fibrous scaffold 40 and/or the fibrous scaffold 81. In some cases, the outer shell could be a rectilinear prism, or an ellipsoid shape as shown in FIG. 10. FIG. 10 shows a number of options for what the inner core may look like. As an example, a construct may have an outer shell 150 and a quarter cubic profile inner core 152. A construct may have an outer shell 154 and a gyroid inner core 156. A construct may have an outer shell 158 and a cubic inner core 160. A construct may have an outer shell 162 and an octet inner core 162. A construct may have an outer shell 164 and a tri-hexagon inner core 166. A construct may have an outer shell 168 and a concentric inner core 170. A construct may have an outer shell 172 and a cross inner core 174. A construct may have an outer shell 176 and a grid inner core 178. A construct may have an outer shell 180 and a cubic subdivision inner core 182. A construct may have an outer shell 184 and a cross-3D inner core 186. A construct may have an outer shell 188 and a triangular inner core 190. These are just examples.

In some cases, the relative orientations of the lattices in the fibrous scaffold 40 and in the fibrous scaffold 81 may be modified to meet the primary axis of loading (the major axis 36). For example, for the fibrous scaffold 40, the lattice could be flipped across two planes so that the linear transvers members (the fibers 48) are aligned with the primary axis of loading, and the woven members serve to reinforce the transverse direction.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A bio-compatible implant adapted for use in repairing soft tissue damage, the bio-compatible implant comprising:
a fibrous scaffold having a length, a width and a thickness;
the fibrous scaffold including a plurality of fibers oriented in three orthogonal directions in a lattice structure.

2. The bio-compatible implant of claim 1, wherein the plurality of fibers comprises a first plurality of fibers that extend along the length of the fibrous scaffold within each of a plurality of layers stacked along the thickness of the fibrous scaffold, each of the first plurality of fibers within each layer following an oscillating wave pattern along the length of the fibrous scaffold defining alternating peaks and troughs in which the fiber alternates between a first direction orthogonal to the length and a second direction orthogonal to the length, the fiber remaining within a single layer.

3. The bio-compatible implant of claim 2, wherein the oscillating wave pattern comprises a sinusoidal wave pattern or a square wave pattern.

4. The bio-compatible implant of claim 2, wherein each fiber of the first plurality of fibers within a layer is laterally offset from neighboring fibers within that layer.

5. The bio-compatible implant of claim 4, wherein each fiber of the first plurality of fibers within a layer overlaps with neighboring fibers in an adjacent layer.

6. The bio-compatible implant of claim 5, wherein each fiber defines peaks that overlap with peaks and troughs that overlap with troughs of neighboring fibers in an adjacent layer.

7. The bio-compatible implant of claim 2, wherein the fibrous scaffold further comprises a second plurality of fibers that extend orthogonally to the first plurality of fibers and extend through multiple layers.

8. The bio-compatible implant of claim 7, wherein some of the second plurality of fibers extend through peaks formed by fibers of the first plurality of fibers within a plurality of layers and some of the second plurality of fibers extend through troughs formed by fibers of the first plurality of fibers within a plurality of layers.

9. The bio-compatible implant of claim 7, wherein at least two fibers of the second plurality of fibers extend through each of the peaks and through each of the troughs.

10. The bio-compatible implant of claim 1, wherein the plurality of fibers each include multiple periods of a loop pattern in which each fiber repeatedly includes a climbing loop, a vertical segment and a straight segment joining the climbing loop and the vertical segment.

11. The bio-compatible implant of claim 10, wherein for each of the plurality of fibers, the vertical segment passes through a climbing loop of another fiber.

12. The bio-compatible implant of claim 10, wherein the straight segment after a climbing loop extends in a direction orthogonal to a direction in which the straight segment before the climbing loop extends.

13. The bio-compatible implant of claim 12, wherein the straight segment after a climbing loop is also offset in a direction orthogonal to the direction in which the straight segment after the climbing loop extends and orthogonal to the direction in which the straight segment before the climbing loop extends.

14. The bio-compatible implant of claim 12, wherein each vertical segment extends in a direction orthogonal to the direction in which the straight segment after the climbing loop extends and orthogonal to the direction in which the straight segment before the climbing loop extends.

15. The bio-compatible implant of claim 1, wherein the fibrous scaffold is formed via embedded bioprinting.

16. A bio-compatible implant adapted for use in repairing soft tissue damage, the bio-compatible implant comprising:
a fibrous scaffold having a length, a width and a thickness;
the fibrous scaffold including a plurality of layers arranged atop each other;
the fibrous scaffold comprising:
a first plurality of fibers that extend along the length of the fibrous scaffold within each of the plurality of layers, each of the first plurality of fibers within each layer following a wave pattern along the length of the fibrous scaffold defining alternating peaks and troughs in which the fiber alternates between a first direction and a second direction, the fiber remaining within a single layer; and
a second plurality of fibers that extend orthogonally to the first plurality of fibers and extend through multiple layers.

17. The bio-compatible implant of claim 16, wherein a tensile force applied along the length of the fibrous scaffold causes fibers of the first plurality of fibers to engage fibers of the second plurality of fibers.

18. The bio-compatible implant of claim 16, wherein the plurality of layers are disposed relative to each other in an anterior-posterior direction.

19. The bio-compatible implant of claim 16, wherein each of the first plurality of fibers extend in a lateral-medial direction while alternating between an inferior direction and a superior direction while alternating to form the peaks and troughs.

20. A bio-compatible implant adapted for use in repairing soft tissue damage, the bio-compatible implant comprising:
a fibrous scaffold comprising a plurality of fibers that each include multiple periods of a loop pattern in which each fiber repeatedly forms a climbing loop, a vertical segment and a straight segment joining the climbing loop and the vertical segment;
wherein for each of the plurality of fibers, the vertical segment passes through a climbing loop of another fiber.
